(19) United States
(12) Patent Application Publication
(10) Pub. No.: US 2025/0009252 A1
(43) Pub. Date: Jan. 9, 2025

(54) ACCURATE DETERMINATION OF RESPIRATORY RATES USING DEPTH-BASED CAMERA SYSTEM

(71) Applicant: Covidien LP, Mansfield, MA (US)

(72) Inventors: Andre Antunes, Edinburgh (GB); Dean Montgomery, Edinburgh (GB); Paul S. Addison, Edinburgh (GB); Philip C. Smit, Hamilton (GB)

(21) Appl. No.: 18/755,304

(22) Filed: Jun. 26, 2024

Related U.S. Application Data
(60) Provisional application No. 63/511,945, filed on Jul. 5, 2023.

(51) Int. Cl. A61B 5/08; A61B 5/00; A61B 5/091

(52) U.S. Cl. CPC A61B 5/0816; A61B 5/0077; A61B 5/091; A61B 5/7239; A61B 5/7257; A61B 5/7264; A61B 5/742; A61B 2560/045

(57) ABSTRACT

Methods for improving the accurate determination of respiratory rate, the respiratory rate being measured or monitored from depth measurements from a non-contact monitoring system. The methods include analyzing a waveform obtained from a respiratory parameter and discarding data that does not meet criteria. The respiratory rate may be obtained from an original waveform, a processed waveform, or from a power spectrum derived from the original or processed waveform.

### CROSS-REFERENCE TO RELATED APPLICATION

The present application claims the benefit of U.S. Provisional Patent Application Ser. No. 63/511,945, filed on Jul. 5, 2023, the entire content of whish is incorporated herein by reference.

### BACKGROUND

Video-based monitoring is a field of patient monitoring that uses a remote video camera to detect physical attributes of the patient. This type of monitoring may also be called “non-contact” monitoring in reference to the remote video sensor, which does not contact the patient. Such non-contact monitoring can be used to determine patient movement, such as arm or leg movement. Non-contact monitoring can also be used to determine a patient's respiratory rate, respiratory volume, pulse and/or heart rate, based on movement of, e.g., the patient's chest.

Smaller movements, however, provide the opportunity for inaccurate measurements, or, for a high probability of inaccurate measures.

### SUMMARY

The present disclosure provides various methods for improving the accurate determination of respiratory rate, the respiratory rate being measured or monitored from depth measurements from a non-contact monitoring system. The methods include analyzing a waveform, such as flow or volume, obtained from a respiratory parameter and discarding data that does not meet criteria. In some embodiments, the waveform may be converted to, e.g., a power spectrum waveform. The waveform, the power spectrum, or a combination of the two can be used determine an accurate respiratory rate. In an embodiment, the features of the waveform are compared to features of the power spectrum and one or a combination of the waveform and the power spectrum are used to determine an accurate respiratory rate. For example, a preliminary respiratory rate is determined from the waveform and from the power spectrum, and dependent on the preliminary respiratory rates, one or both of the waveform and the power spectrum are used to determine an accurate respiratory rate.

The disclosure includes determining a respiratory parameter of the subject with a non-contact monitoring system using depth measurements, the respiratory parameter being one or more of respiratory flow, respiratory volume, and respiratory rate, and then dependent on the respiratory parameter, producing one or both of a waveform of the respiratory parameter over a time period or a power spectrum waveform from the respiratory parameter over the time period. From one or both of these waveforms, an accurate respiratory rate can be determined.

One particular embodiment described herein is a non-contact monitoring system for determining a respiratory rate of a subject, the system comprising a depth-sensing camera, a display, and a processor and a memory storing instructions therein. The instructions, when executed by the processor, cause the processor to determine a tidal volume of the subject with the non-contact monitoring system using depth measurements, calculate a respiratory flow waveform from the respiratory volume over a time period, determine a flow-based respiratory rate from the respiratory flow waveform, calculate a power spectrum from the respiratory flow waveform and determine a spectrum-based respiratory rate from the power spectrum, select between the flow-based respiratory rate and the spectrum-based respiratory rate based on a respiratory rate threshold, and show the selected respiratory rate on the display.

Another particular embodiment described herein is a method for determining an accurate respiratory rate, the method including determining a respiratory volume of the subject with the non-contact monitoring system using depth measurements, calculating a respiratory flow waveform from the respiratory volume over a time period, determine a flow-based respiratory rate from the respiratory flow waveform, calculating a power spectrum from the respiratory flow waveform and determine a spectrum-based respiratory rate from the power spectrum, and selecting between the flow-based respiratory rate and the spectrum-based respiratory rate based on a respiratory rate threshold.

Another particular embodiment described herein is a method that includes determining a respiratory volume of the subject with the non-contact monitoring system using depth measurements, calculating a respiratory flow waveform from the tidal volume over a time period, determining a respiratory rate from the respiratory flow waveform, dependent on the respiratory rate from the respiratory flow waveform being above a threshold (e.g., about 30 breaths/minute, or, about 30 to 35 breaths/minute), calculating a power spectrum from the respiratory flow waveform and determining a respiratory rate from the power spectrum, and dependent on the respiratory rate from the power spectrum being determined, displaying the respiratory rate from the power spectrum to a user, otherwise, displaying to the user the respiratory rate from the respiratory flow waveform.

Yet another particular embodiment described herein is a non-contact monitoring system having a processor configured to determine a preliminary respiratory rate and a tidal volume of the subject with the non-contact monitoring system using depth measurements, calculate a respiratory flow from the tidal volume and form a waveform of the respiratory flow over a time period, dependent on the preliminary respiratory rate being above the threshold (e.g., about 30 to 35 breaths/minute), calculate a power spectrum from the waveform of the respiratory flow and determine a respiratory rate from the power spectrum, dependent on the preliminary respiratory rate being below the threshold, and determine the respiratory rate from the waveform of the respiratory flow.

An alternate embodiment described herein has a processor configured to determine preliminary respiratory rate and a respiratory volume of the subject with the non-contact monitoring system using depth measurements, calculate a respiratory flow waveform from the respiratory volume over a time period, dependent on the preliminary respiratory rate being below a threshold, determine a respiratory rate from the respiratory flow waveform, dependent on the preliminary respiratory rate being above a threshold (e.g., about 30 to 35 breaths/minute), calculate a power spectrum from the respiratory flow waveform and determine a respiratory rate from the power spectrum, and provide to a user at least one of the respiratory rate from the respiratory flow waveform and the respiratory rate from the power spectrum.

Other embodiments are also described and recited herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

These and other aspects of the technology described herein will be apparent after consideration of the Detailed Description and Drawing herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWING

FIG. 1 is a schematic diagram of an example non-contact monitoring system positioned in relation to a patient being monitored.

FIG. 2 is a schematic diagram of another example non-contact monitoring system.

FIG. 3 is a block diagram of a non-contact monitoring system including a computing device, a server, and an image capture device according to various embodiments described herein.

FIG. 4 is a perspective view of another example non-contact monitoring system.

FIG. 5 is a perspective view of yet another non-contact monitoring system positioned in relation to a bed.

FIG. 6 is a side view of yet another non-contact monitoring system positioned in relation to a bed.

FIG. 7 is a graphical representation showing the relation of respiratory volume and respiratory flow.

FIGS. 8A and 8B are a graphical representations of example techniques for validating a breath for respiratory rate.

FIG. 9 is another graphical representation of an example technique for validating a breath for respiratory rate.

FIG. 10 is another graphical representation of an example technique for validating a breath for respiratory rate.

FIG. 11 is another graphical representation of an example technique for validating a breath for respiratory rate.

FIG. 12 is another graphical representation of an example technique for validating a breath for respiratory rate.

FIG. 13 is another graphical representation of an example technique for validating a breath for respiratory rate.

FIG. 14 is a step-wise flow chart of a method for validating a breath for respiratory rate.

FIG. 15 is graphical representations of regions invalidated around a motion flag.

FIG. 16 is a graphical representation of invalid region extension to the local minimum of a waveform.

FIG. 17 is a graphical representation of a waveform with start and end regions excluded for the first minima in the waveform.

FIG. 18 is graphical representations of a linear compensation applied to a waveform.

FIG. 19 is graphical representations of linear frequency scaling applied to a power spectrum.

FIG. 20 is graphical representations of derivative-enhanced transformation of a power spectrum.

FIG. 21 is a graphical image of a wavelet time-frequency power spectrum.

FIG. 22 is a graphical representation comparing respiratory rate as determined by a non-contact depth sensing system to a respiratory rate determined by a Reference EtCO₂ monitor.

FIG. 23 is a second graphical representation comparing respiratory rate as determined by a non-contact depth sensing system to a respiratory rate determined by a Reference EtCO₂ monitor.

### DETAILED DESCRIPTION

One of the most common vital signs measured in a clinical setting is respiratory rate (RR). A significant change in RR can often be an early indication of a major complication such as respiratory tract infection, respiratory depression associated with opioid consumption, anesthesia and/or sedation, as well as respiratory failure. As described above, the present disclosure is directed to monitoring a subject (e.g., a patient) while resting or sleeping with a depth sensing, non-contact monitoring system to determine respiratory rate (RR). The disclosure provides various techniques to improve the accurate determination of the respiratory rate.

The monitoring of the subject is accomplished with a non-contact monitoring system that uses a video signal of the subject, identifying physiologically relevant areas within the video image (such as the subject's head, face, neck, arms, legs, or torso). The system may use vision-based artificial intelligence (AI) methods to learn to better identify the relevant areas. The system can be used in a medical or commercial setting, such as in a hospital or a clinic, or in a residential setting.

With non-contact monitoring systems, signals representative of the topography and movement of the topography, of the subject, are detected by a camera or camera system that views but does not contact the subject. The camera or camera system may utilize any or all of depth signals, color signals (e.g., RGB signals), and IR signals. With appropriate selection and filtering of the signals detected by the camera, the physiologic contribution by each of the detected signals can be isolated and measured.

The non-contact monitoring systems receives a video signal from the subject and from that extract a distance or depth signal from the relevant area to provide a topographical map from the depth signal; the systems may also determine any movement or motion from the depth signal. The systems can also receive a second signal, a light intensity signal reflected from the subject, and from the reflected light intensity signal calculate a depth or distance and also a movement or motion. In some embodiments, the light intensity signal is a reflection of a pattern or feature (e.g., using visible color or infrared) projected onto the subject, such as by a projector.

The depth sensing feature of the systems provides a measurement of the distance or depth between the detection system and the subject. One or two video cameras may be used to determine the depth, and change in depth, from the system to the subject. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system may have difficulty resolving the perspective differences. By including an image projector to project features (e.g., in the form of dots, pixels, etc., visual or IR) onto the scene, this projected feature can be monitored over time to produce an estimate of location and any change in location of an object.

Described herein are two general methodologies to determining an accurate respiratory rate from a depth signal. One methodology uses a flow signal, which is derived from the volume, to detect the breaths and thus determine the respiratory rate. The flow signal is used because it is easier to detect zero crossings in the flow signal than in a volume signal. Another methodology uses the frequency domain, or power spectrum, to detect the dominant frequency in the depth signal.

Patients breathe in all kinds of different ways-deep, shallow, fast, slow, some with different ratios of inhalation to exhalation. When a patient's respiratory rate is lower, the flow signal methodology can be readily used to accurately determine the respiratory rate. At low respiratory rates, the volume tends to be relatively high; high volumes are indicated by large changes in depth measurements, which can be readily detected over noise in the depth signal.

With lower breathing rates, the inhalation:exhalation ratio can significantly vary for an individual at a time, e.g., it may be 1:1 for a few minutes and 1:4 soon thereafter. Such an irregular breathing pattern does not produce a clear sinusoidal waveform, so the frequency domain or power spectrum methodology is not as helpful.

The frequency domain or power spectrum methodology can be readily used to accurately determine the respiratory rate when the rate is fast. Fast and very fast breathing tend to have a clear frequency, close to a sinusoidal waveform, having essentially a 1:1 inhalation:exhalation ratio, which is prominent in a frequency domain (the frequency of the sinusoid). But because the respiratory volumes may be low for a fast respiratory rate, the respiratory rate may be harder to detect against any background noise. The frequency domain methodology may not be suitable for all examples, as the methodology does not provide accurate results for a very noisy depth signal. Additionally, if the respiratory rate does not have a regular rate, a clear frequency signal may not be obtained.

At least for these reasons, in some embodiments, if the respiratory rate is at or above a threshold (e.g., high or fast, e.g., above about 30 breaths/minute), the frequency signal is used to accurately determine the respiratory rate, whereas if the respiratory rate is at or below the threshold (e.g., low or slow), the respiratory volume is used to accurately determine the respiratory rate. In some embodiments, it may be desired to determine the accurate respiratory rate by both methodologies and combine the determined rate, particularly when the respiratory rate is close to the threshold, e.g., within +/−5% (that is, 95% to 105% of the threshold), or within +/−10% (that is, 90% to 110% of the threshold), in some embodiments, even within as much as +/−25% (that is, 75% to 125% of the threshold).

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

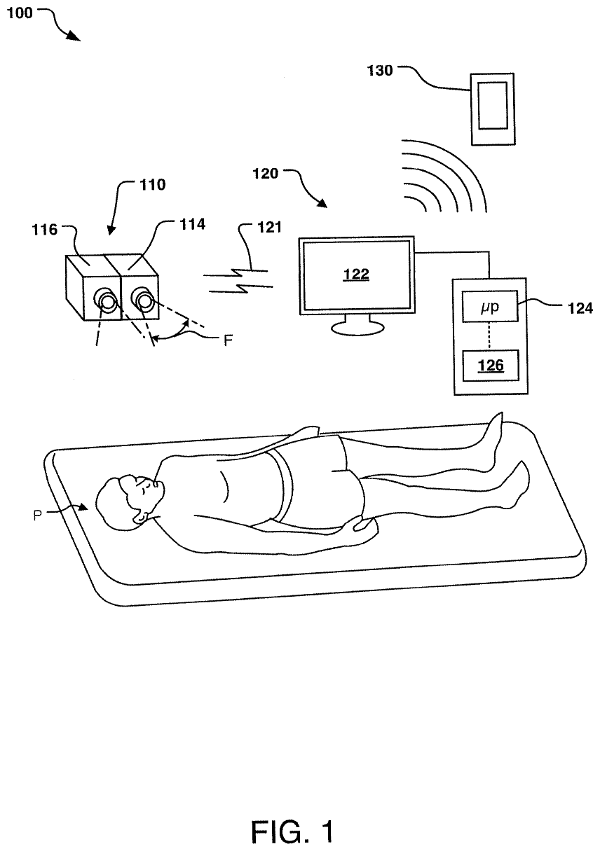

FIG. 1 shows a non-contact subject monitoring system 100 and a subject P. The system 100 includes a non-contact detector system 110 placed remote from the subject P. In this embodiment, the detector system 110 includes a camera system 114, particularly, a camera that includes an infrared (IR) detection feature. The camera system 114 may be a depth sensing camera system, such as a Kinect camera from Microsoft Corp. (Redmond, Washington) or a RealSense™ D415, D435 or D455 camera from Intel Corp. (Santa Clara, California).

The camera system 114 may operate at a set frame rate, which is the number of image frames taken per second (or other time period). Example frame rates include 15, 20, 30, 40, 50, or 60 frames per second, greater than 60 frames per second, or other values between those. Frame rates of 15-30 frames per second produce useful signals, though frame rates above 100 or 120 frames per second are helpful in avoiding aliasing with light flicker (for artificial lights having frequencies around 50 or 60 Hz).

The camera system 114 is remote from the subject P, in that it is spaced apart from and does not physically contact the subject P. The camera system 114 may be positioned in close proximity to or attached to a bed. The camera system 114 has a field of view F that encompasses at least a portion of the subject P.

The field of view F is selected to be at least the upper torso of the subject. However, as it is common for young children and infants to move within the confines of their crib, bed or other sleeping area, the entire area potentially occupied by the subject P (e.g., a crib) may be the field of view F.

The camera system 114 includes a depth sensing camera that can detect a distance between the camera system 114 and objects in its field of view F. Such information can be used to determine that the subject is within the field of view of the camera system 114 and determine a region of interest (ROI) to monitor on the subject. The ROI may be the entire field of view F or may be less than the entire field of view F. Once an ROI is identified, the distance to the desired feature is determined and the desired measurement(s) can be made.

The ROI is monitored over time. The distance from the ROI on the subject P to the camera system 114 is measured by the system 100. Generally, the camera system 114 detects a distance between the camera system 114 and the surface within the ROI. With this distance, the system 100 can determine the location on the subject (e.g., the chest) and the change in location, e.g., due to the subject moving, e.g., the rise and fall of the chest associated with breathing.

In some embodiments, the system 100 determines a skeleton outline of the subject P to identify a point or points from which to extrapolate the ROI. For example, a skeleton may be used to find a center point of a chest, shoulder points, waist points, hands, head, and/or any other points on a body. These points can be used to determine the ROI. In other embodiments, instead of using a skeleton, other points are used to establish an ROI. For example, a face may be recognized, and a torso and waist area inferred in proportion and spatial relation to the face.

In another example, the subject P may wear a specially configured piece of clothing that identifies points on the body such as the torso or the arms. The system 100 may identify those points by identifying the indicating feature of the clothing. Such identifying features could be a visually encoded message (e.g., bar code, QR code, etc.), or a brightly colored shape that contrasts with the rest of the subject's clothing, etc. In some embodiments, a piece of clothing worn by the subject may have a grid or other identifiable pattern on it to aid in recognition of the subject and/or their movement. In some embodiments, the identifying feature may be stuck on the clothing using a fastening mechanism such as adhesive, a pin, etc., or stuck directly on the subject's skin, such as by adhesive. For example, a small sticker or other indicator may be placed on a subject's hands that can be easily identified from an image captured by a camera.

In some embodiments, the system 100 may receive a user input to identify a starting point for defining an ROI. For example, an image may be reproduced on an interface, allowing a user of the interface to select a point on the subject from which the ROI can be determined (such as a point on the chest). Other methods for identifying a subject, points on the subject, and defining an ROI may also be used.

However, if the ROI is essentially featureless (e.g., a smooth surface with a monochromatic color, such as a blanket or sheet covering the subject P), then the camera system 114 may have difficulty resolving the perspective differences. To address this, the system 100 can include a projector 116 to project individual features (e.g., dots, crosses or Xs, lines, individual pixels, etc.) onto objects in the ROI; the features may be visible light, UV light, infrared (IR) light, etc. The projector may be part of the detector system 110 or the overall system 100.

The projector 116 generates a sequence of features over time on the ROI from which is monitored and measured the reflected light intensity. A measure of the amount, color, or brightness of light within all or a portion of the reflected feature over time is referred to as a light intensity signal. The camera system 114 detects the features from which this light intensity signal is determined. In an embodiment, each visible image projected by the projector 116 includes a two-dimensional array or grid of pixels, and each pixel may include three color components—for example, red, green, and blue (RGB). A measure of one or more color components of one or more pixels over time is referred to as a “pixel signal,” which is a type of light intensity signal. In another embodiment, when the projector 116 projects an IR feature, which is not visible to a human eye, the camera system 114 includes an infrared (IR) sensing feature. In another embodiment, the projector 116 projects a UV feature. In yet other embodiments, other modalities including millimeter-wave, hyper-spectral, etc., may be used.

The projector 116 may alternately or additionally project a featureless intensity pattern (e.g., a homogeneous, a gradient or any other pattern that does not necessarily have distinct features, or a pattern of random intensities). In some embodiments, the projector 116, or more than one projector, can project a combination of a feature-rich pattern and featureless patterns on to the ROI.

The light intensity of the image reflected by the subject surface is detected by the detector system 110.

The measurements (e.g., one or more of depth signal, RGB reflection, light intensity) are sent to a computing device 120 through a wired or wireless connection 121. The computing device 120 includes a display 122, a processor 124, and hardware memory 126 for storing software and computer instructions. Sequential image frames of the subject P are recorded by the video camera system 114 and sent to the computing device 120 for analysis by the processor 124. The display 122 may be remote from the computing device 120, such as a video screen positioned separately from the processor and memory. Other embodiments of the computing device 120 may have different, fewer, or additional components than shown in FIG. 1. In some embodiments, the computing device may be a server. In other embodiments, the computing device of FIG. 1 may be connected to a server. The captured images (e.g., still images or video) can be processed or analyzed at the computing device and/or at the server to create a topographical map or image to identify the subject P and any other objects with the ROI.

In some embodiments, the computing device 120 is operably connected (e.g., wirelessly, via WiFi connectivity, cellular signal, Bluetooth™ connectivity, etc.) to a remote device 130 such as a smart phone, tablet, or merely a screen. The remote device 130 can be remote from the computing device 120 and the subject P, for example, in an adjacent or nearby room. The computing device 120 may send a video feed to the remote device 130, showing e.g., the subject P and/or the field of view F. Additionally or alternately, the computing device 120 may send instructions to the remote device 130 to advise a clinician or caregiver of the status of the subject P.

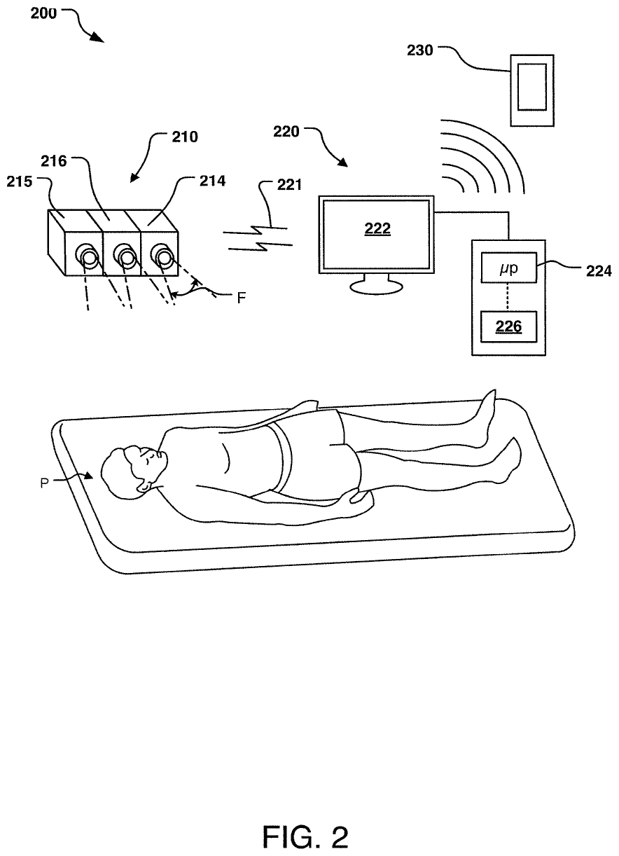

FIG. 2 shows another non-contact subject monitoring system 200 and a subject P. The system 200 includes a non-contact detector 210 placed remote from the subject P. In this embodiment, the detector 210 includes a first camera 214 and a second camera 215, at least one of which includes an infrared (IR) camera feature. The cameras 214, 215 are positioned so that their ROIs at least intersect, in some embodiments, closely overlap. The detector 210 also includes an IR projector 216, which projects individual features (e.g., dots, crosses or Xs, lines, or a featureless pattern, or a combination thereof etc.) onto the subject P in the ROI. The projector 216 can be separate from the detector 210 or integral with the detector 210, as shown in FIG. 2. In some embodiments, more than one projector 216 can be used. Both cameras 214, 215 are aimed to have features projected by the projector 216 to be in their ROI. The cameras 214, 215 and the projector 216 are remote from the subject P, in that they are spaced apart from and do not contact the subject P. In this implementation, the projector 216 is physically positioned between the cameras 214, 215, whereas in other embodiments it may not be so.

The distance from the ROI to the cameras 214, 215 is measured by the system 200. Generally, the cameras 214, 215 detect a distance between the cameras 214, 215 and the projected features on a surface within the ROI. The light from the projector 216 hitting the surface is scattered/diffused in all directions; the diffusion pattern depends on the reflective and scattering properties of the surface. The cameras 214, 215 also detect the light intensity of the projected individual features in their ROIs. From the distance and the light intensity, the presence of the subject P and any objects are monitored, as well as any movement of the subject P or objects.

The detected images, diffusion measurements and/or reflection pattern are sent to a computing device 220 through a wired or wireless connection 221. The computing device 220 includes a display 222, a processor 224, and hardware memory 226 for storing software and computer instructions. The display 222 may be remote from the computing device 220, such as a video screen positioned separately from the processor and memory. In other embodiments, the computing device of FIG. 2 may be connected to a server. The captured images (e.g., still images or video) can be processed or analyzed at the computing device and/or at the server to create a topographical map or image to identify the subject P and any other objects with the ROI.

In some embodiments, the computing device 220 is operably connected (e.g., wirelessly, via WiFi connectivity, cellular signal, Bluetooth™ connectivity, etc.) to a remote device 230 such as a smart phone, tablet, or merely a screen. The remote device 230 can be remote from the computing device 220 and the subject P, for example, in an adjacent or nearby room. The computing device 220 may send a video feed to the remote device 230, showing, e.g., the subject P and/or the field of view F.

The computing device 120, 220 has an appropriate memory, processor, and software or other program to evaluate the ROI image, identify features of the subject, and maintain a database. The computing device 120, 220 can be trained with vision-based artificial intelligence (AI) methods to learn to identify particular physical features of the subject. The computing device 120, 220 can be trained using any standard AI model and standard methods, e.g., utilizing numerous data points to create a dataset of images.

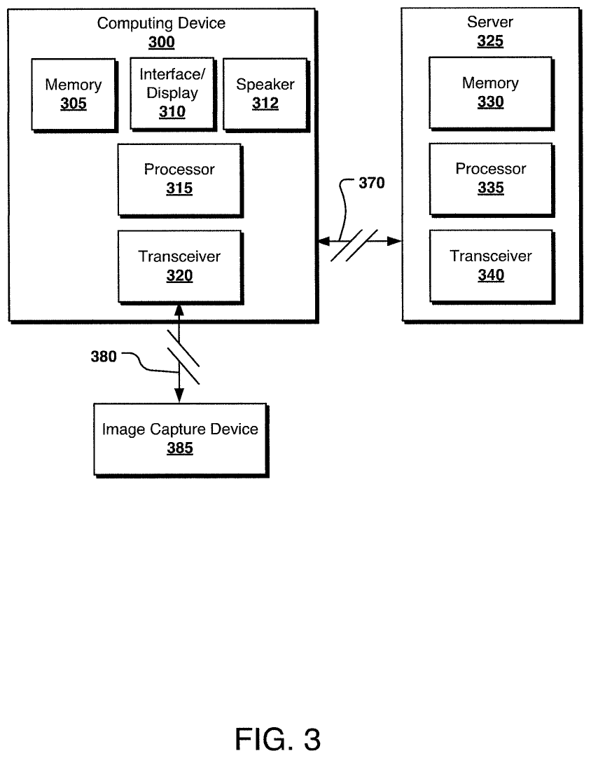

FIG. 3 is a block diagram illustrating a system including a computing device 300, a server 325, and an image capture device 385 (e.g., a camera, e.g., the camera system 114 or cameras 214, 215). In various embodiments, fewer, additional and/or different components may be used in the system.

The computing device 300 includes a processor 315 that is coupled to a memory 305. The processor 315 can store and recall data and applications in the memory 305, including applications that process information and send commands/signals according to any of the methods disclosed herein. The processor 315 may also display objects, applications, data, etc. on an interface/display 310 and/or provide an audible alert via a speaker 312. The processor 315 may also or alternately receive inputs through the interface/display 310. The processor 315 is also coupled to a transceiver 320. With this configuration, the processor 315, and subsequently the computing device 300, can communicate with other devices, such as the server 325 through a connection 370 and the image capture device 385 through a connection 380. For example, the computing device 300 may send to the server 325 information determined about a subject from images captured by the image capture device 385, such as depth information of a subject or object in an image.

The server 325 also includes a processor 335 that is coupled to a memory 330 and to a transceiver 340. The processor 335 can store and recall data and applications in the memory 330. With this configuration, the processor 335, and subsequently the server 325, can communicate with other devices, such as the computing device 300 through the connection 370.

The computing device 300 may be, e.g., the computing device 120 of FIG. 1 or the computing device 220 of FIG. 2. Accordingly, the computing device 300 may be located remotely from the image capture device 385, or it may be local and close to the image capture device 385 (e.g., in the same room). The processor 315 of the computing device 300 may perform any or all of the various steps disclosed herein. In other embodiments, the steps may be performed on a processor 335 of the server 325. In some embodiments, the various steps and methods disclosed herein may be performed by both of the processors 315 and 335. In some embodiments, certain steps may be performed by the processor 315 while others are performed by the processor 335. In some embodiments, information determined by the processor 315 may be sent to the server 325 for storage and/or further processing.

The devices shown in the illustrative embodiment may be utilized in various ways. For example, either or both of the connections 370, 380 may be varied. For example, either or both the connections 370, 380 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 370, 380 may be a dock where one device may plug into another device. As another example, one or both of the connections 370, 380 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 370, 380 may represent several different computing devices and network components that allow the various devices to communicate through the internet, either through a hard-wired or wireless connection. One or both of the connections 470, 480 may also be a combination of several modes of connection.

The configuration of the devices in FIG. 3 is merely one physical system on which the disclosed embodiments may be executed. Other configurations of the devices shown may exist to practice the disclosed embodiments. Further, configurations of additional or fewer devices than the ones shown in FIG. 3 may exist to practice the disclosed embodiments. Additionally, the devices shown in FIG. 3 may be combined to allow for fewer devices than shown or separated such that more than the three devices exist in a system. It will be appreciated that many various combinations of computing devices may execute the methods and systems disclosed herein. Examples of such computing devices may include other types of infrared cameras/detectors, night vision cameras/detectors, other types of cameras, radio frequency transmitters/receivers, smart phones, personal computers, servers, laptop computers, tablets, RFID enabled devices, or any combinations of such devices.

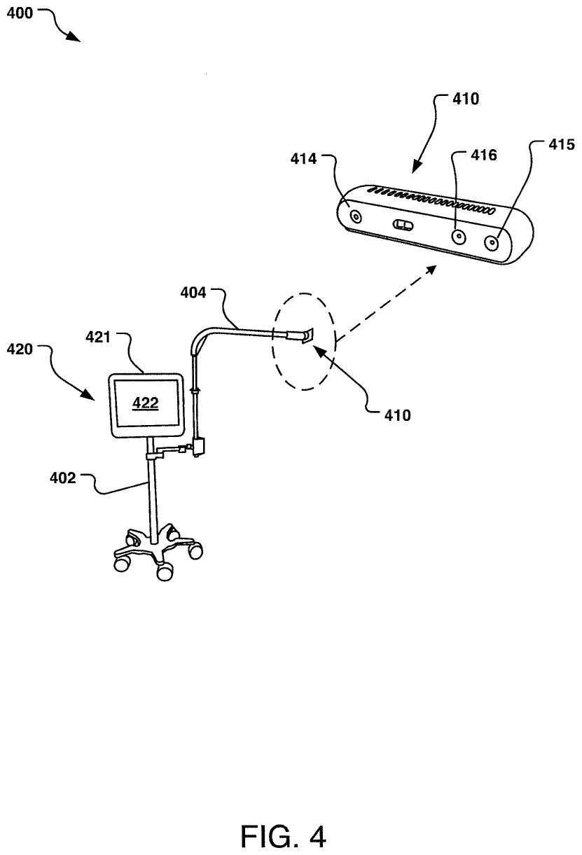

Alternate configurations of non-contact monitoring systems are shown in FIGS. 4, 5 and 6.

FIG. 4 shows a portable non-contact subject monitoring system 400 that includes a non-contact detector 410 and a computing device 420. In this embodiment, the non-contact detector 410 and the computing device 420 are generally fixed in relation to each other and the system 400 is readily moveable in relation to the subject to be monitored. The detector 410 and the computing device 420 are supported on a trolley or stand 402, with the detector 410 on an arm 404 that is pivotable in relation to the stand 402 as well as adjustable in height. The system 400 can be readily moved and positioned where desired.

The detector 410 includes a first camera 414 and a second camera 415, at least one of which includes an infrared (IR) camera feature. The detector 410 also includes an IR projector 416, which projects individual features (e.g., dots, crosses or Xs, lines, or a featureless pattern, or a combination thereof etc.).

The detector 410 may be wired or wireless connected to the computing device 420. The computing device 420 includes a housing 421 with a touch screen display 422, a processor (not seen), and hardware memory (not seen) for storing software and computer instructions.

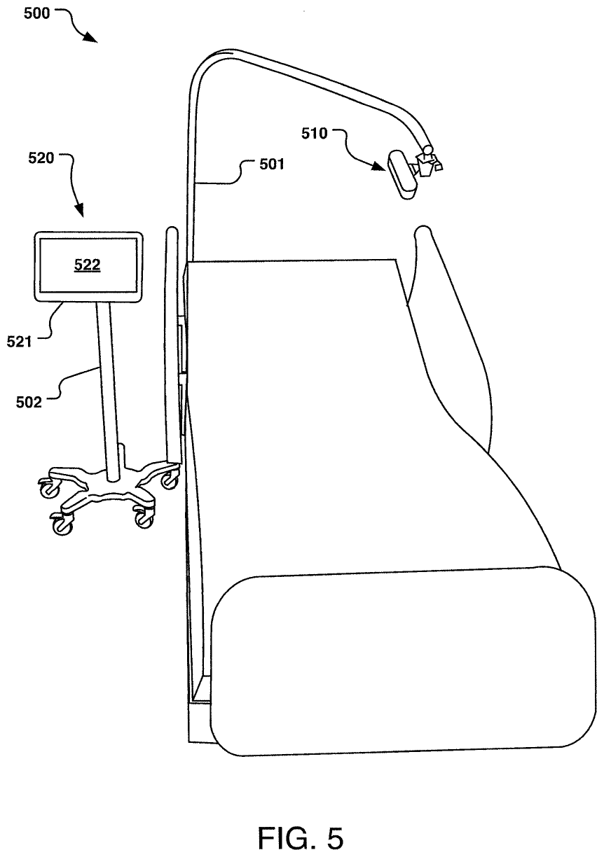

FIG. 5 shows a semi-portable non-contact subject monitoring system 500 that includes a non-contact detector 510 and a computing device 520. In this embodiment, the non-contact detector 510 is in a fixed relation to the subject to be monitored and the computing device 520 is readily moveable in relation to the subject.

The detector 510 is supported on an arm 501 that is attached to a bed, in this embodiment, a hospital bed, although the detector 510 and the arm 501 can be attached to a crib, a bassinette, an incubator, an isolette, or other bed-type structure. In some embodiments, the arm 501 is pivotable in relation to the bed as well as adjustable in height to provide for proper positioning of the detector 510 in relation to the subject.

The detector 510 may be wired or wireless connected to the computing device 520, which is supported on a moveable trolley or stand 502. The computing device 520 includes a housing 521 with a touch screen display 522, a processor (not seen), and hardware memory (not seen) for storing software and computer instructions.

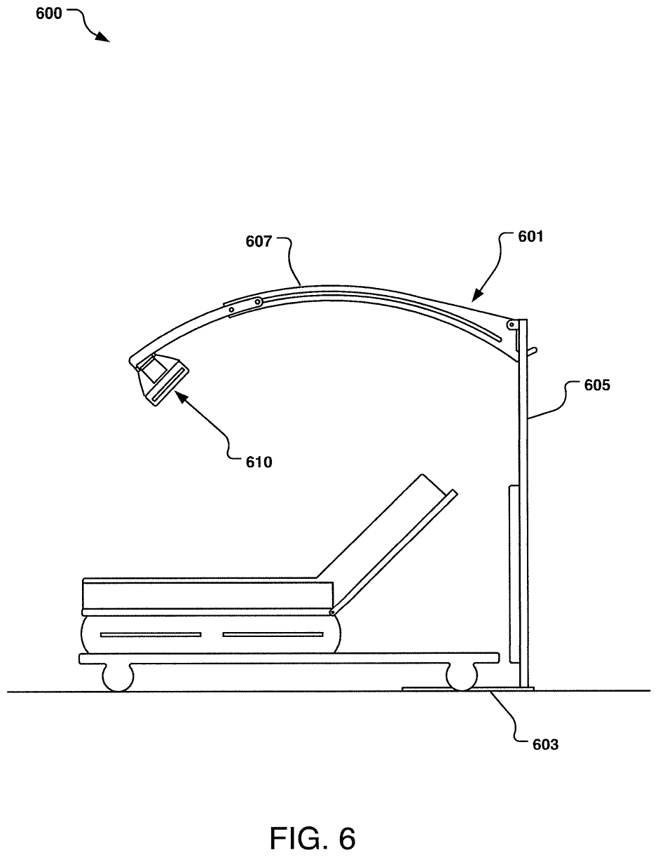

FIG. 6 shows a non-portable non-contact subject monitoring system 600 that includes a non-contact detector 610 and a computing device (not seen in FIG. 6). In this embodiment, at least the non-contact detector 610 is generally fixed in a location, configured to have the subject to be monitored moved into the appropriate position to be monitored.

The detector 610 is supported on a stand 601 that is free standing, the stand having a base 603, a frame 605, and a gantry 607. The gantry 607 may have an adjustable height, e.g., movable vertically along the frame 605, and may be pivotable, extendible and/or retractable in relation to the frame 605. The stand 601 is shaped and sized to allow a bed or bed-type structure to be moved (e.g., rolled) under the detector 610.

The non-contact monitoring systems and methods of this disclosure utilize depth (distance) information between the camera(s) and a subject to determine respiratory rate (RR) by identifying breaths, e.g., in the respiratory volume waveform derived by integrating depth information over a spatial region where respiration occurs. The following techniques can be used for a range of respiratory rates, for example, 4 to 40 breaths per minute (brpm) or 1 to 60 brpm, although different techniques may be more suitable that others depending on the respiratory rate.

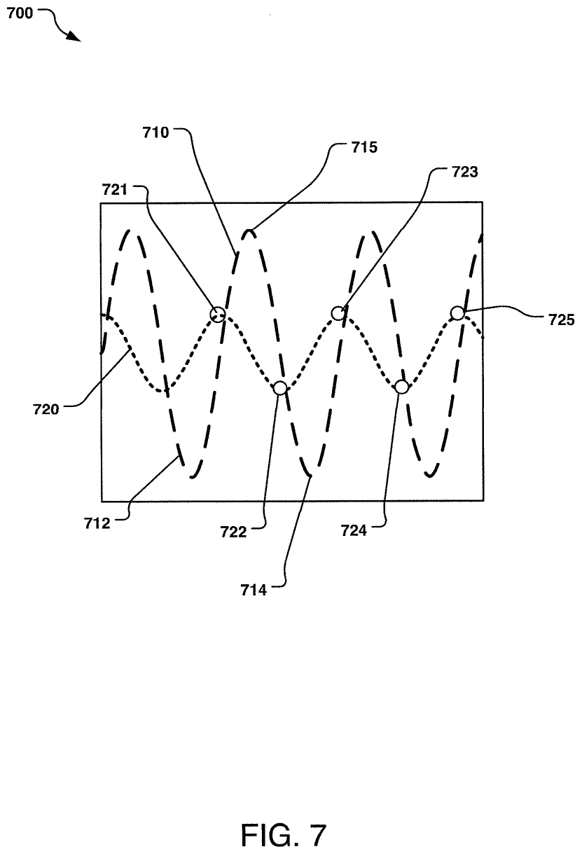

FIG. 7 shows a typical RR waveform from a non-contact monitoring system in a graph 700. The graph 700 shows a volume signal 710 and a flow signal 720 over time. The volume signal 710 has at least one peak, in this figure peak 715, and at least one trough, in this figure, troughs 712, 714. The flow signal 720 has at least one peak, in this figure, peaks 721, 723, 725, and at least one trough, in this figure, troughs 722, 724. A peak 715 in the volume signal 710 occurs between a flow peak 721 and a flow trough 722.

Breaths can be identified in the respiratory volume signal 710 by evaluating two minima, one to the left and one to the right of each maximum or peak, e.g., volume peak 715. The minimum on the left, trough 712, corresponds to the start of inhalation (and the start of the breath). The maximum or peak 715 in the volume signal 710 corresponds to the end of inhalation (and start of exhalation). The minimum on the right, trough 714, corresponds to the end of exhalation (and the end of the breath). Validation or discarding of certain flow peaks/troughs can be used to avoid incorrect breath detection in the volume signal 710, to thus provide accurate respiratory rate. Discussed below are numerous techniques for providing accurate respiratory rate.

Although flow rate, which is the derivative of the respiratory volume or respiratory volume, is used in the discussion herein to improve respiratory rate accuracy, in other embodiments, the same or similar techniques could be used with the volume signal. The derived flow signal provides an advantage to perform the identification and validation of breaths relative to the depth volume signal; the flow signal is naturally centered around zero and does not contain significant drifts. As the flow is the derivative of the volume, a peak (local maximum) in volume must occur between a peak in flow and a trough in flow (FIG. 7). This property allows the use of flow information to discard peaks that are not physiologically plausible (e.g., due to noise, motion, or other confounders).

To perform a validation that the signal actually represents a breath, one or more checks may be performed to identify potentially questionable signals. Questionable signals can then be discounted and removed from the respiratory rate calculation.

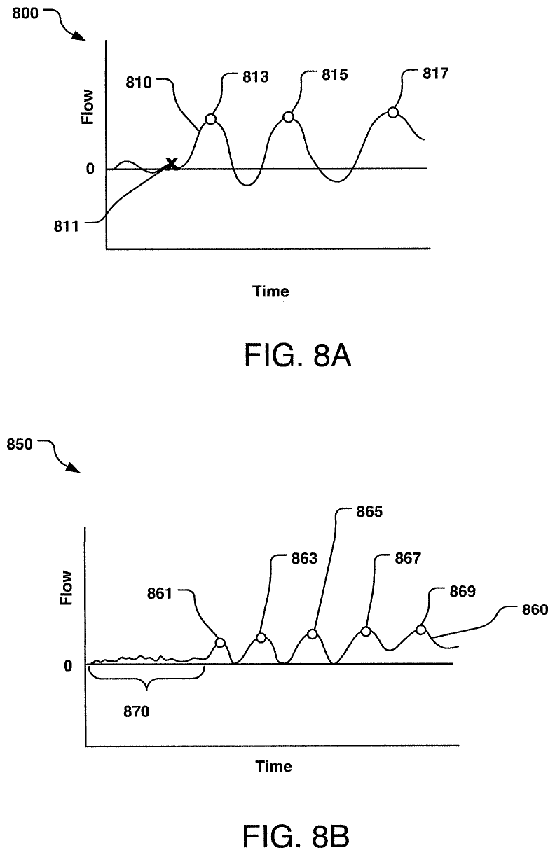

In one validation technique (aka, “low flow before peak”), if the flow that precedes a flow peak is consistently low, then the flow peak will not be considered for the evaluation of a breath. FIG. 8A illustrates a graph 800 having a flow signal 810 over time, the flow signal 810 at least having peaks 811, 813, 815, 817. The peak 811 is significantly lower than the peaks 813, 815, 815, particularly lower than the peak 813. Thus, the peak 811 is not considered for the respiratory rate calculation. In some embodiments, the low peak (e.g., the peak 811) is at least 25%, 50% or 75% lower than the subsequent peak (e.g., peak 813).

A similar technique can be applied to a power or power spectrum signal, the power signal being the squared magnitude of the Fourier transform of the flow signal. A peak or other power value can be discarded if the value of the signal immediately prior to that peak is less than a predetermined value for a predetermined period of time. Similar techniques can be applied to other measures of the flow signal magnitude.

Referring to FIG. 8B, a graph 850 has a flow signal 860 with multiple distinct peaks 861, 863, 865, 867, 869. Any peak that is preceded by a peak less than a predetermined value (e.g., less than a quantitative amount, or, e.g., less than a percentage of the subsequent peak) is discarded. In FIG. 8B, the portion of the signal 860 indicated as in a region 870 is less than a predetermined amount, thus, the peak 861, which is immediately subsequent to the region 870, is discarded from the respiratory rate calculation. As a specific nonlimiting example, if the mean absolute value of the flow in the 5 seconds prior to a peak is less than 10 mL/s (or some other value), that peak is discarded.

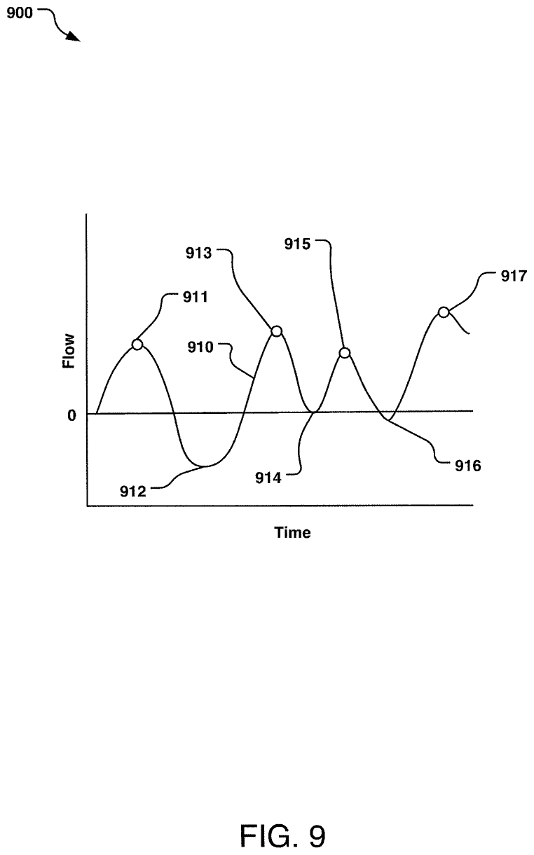

In another validation technique (aka, “low flow at minimum between peaks”), if the minimum of the flow signal between two flow peaks is not sufficiently low, the smaller of the two peaks may be considered invalid and be discarded. Turning to FIG. 9, a graph 900 has a flow signal 910 having peaks 911, 913, 915, 917 with troughs 912, 914, 916 respectively therebetween. The trough 914 is not sufficiently low, e.g., at least 10% of the higher of the surrounding peaks, or at least, for example, 20% lower than the subsequent peak 915, and thus the peak 915 is discarded from the respiratory rate calculation. As a specific nonlimiting example, if a first peak has a flow of 20 mL/s and a second peak has a flow of 12 mL/s, a predetermined minimum (trough) is set at least −2 mL/s. In this example, a minimum flow (trough) of −1 mL/s is not sufficiently low, thus the second peak (12 mL/s) is discarded. As a variation, the lower of the two peaks can be discarded. As an alternate specific nonlimiting example, if a first peak has a flow of 12 mL/s and a second peak has a flow of 20 mL/s, and a predetermined minimum is set at at least −2 mL/s, a minimum flow (trough) of −1 mL/s is not sufficiently low, and thus the lower peak (12 mL/s) is discarded.

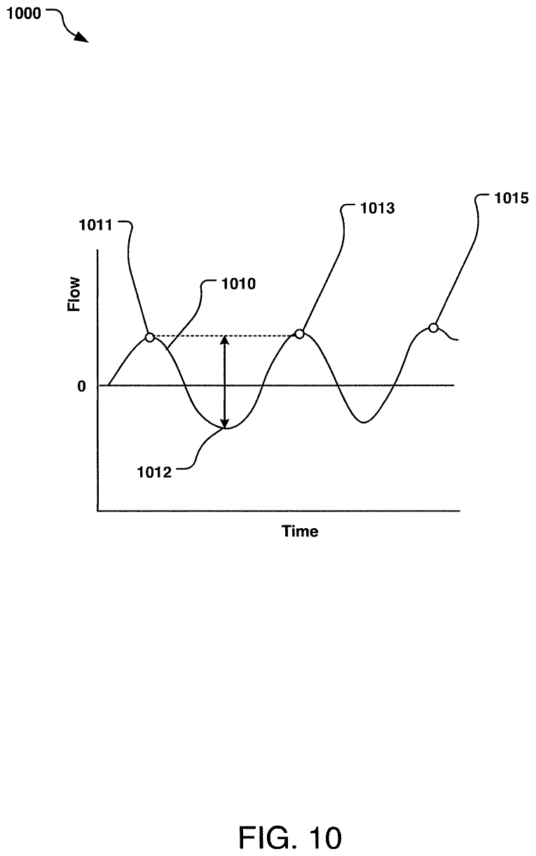

In another validation technique (aka, “flow range between peaks”), if the range of the flow between two consecutive peaks (i.e., the difference between the maximum value and the minimum value in the flow signal between the two peaks) is not within a predetermined threshold, the smallest of the two peaks may be considered invalid and be discarded. Turning to FIG. 10, a graph 1000 has a flow signal 1010 having peaks 1011, 1013, 1015 with a trough 1012 between the peaks 1011 and 1013. If the range between, e.g., peaks 1011 and 1013 and the trough 1012 does not meet the threshold, the smaller of peaks 1011, 1013 is discarded. As a specific nonlimiting example, the difference between the higher of two peaks and the trough therebetween is between 10 mL/s and 100 mL/s.

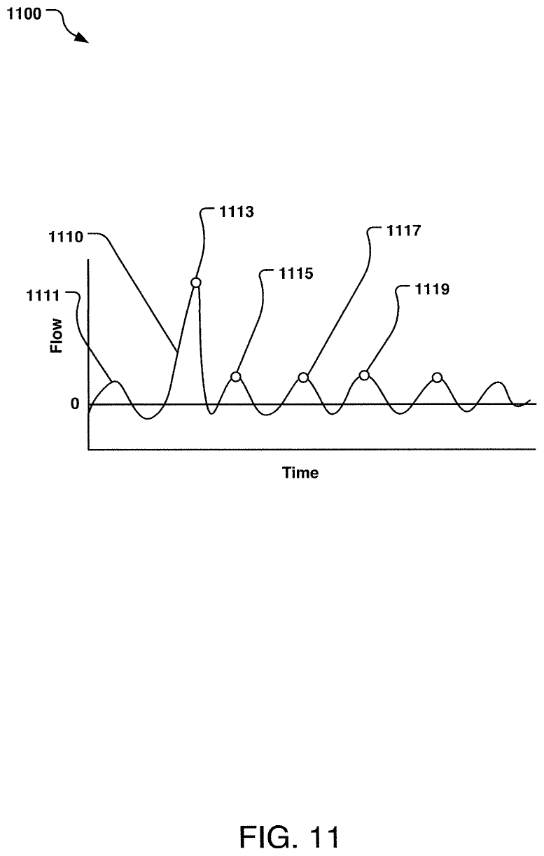

In another validation technique (aka, “magnitude of previous peak”), the magnitude of the peaks in the flow signal that precede a peak under evaluation are examined to decide whether the peak should be validated or discarded. If the magnitude of the preceding peak (or a number of several peaks) that precede the peak are significantly larger or above a certain threshold, the current peak may be invalidated, as well as the increased peak. Turning to FIG. 11, a graph 1100 has a flow signal 1110 having (identified) peaks 1111, 1113, 1115, 1117, 1119. Peak 1113 is significantly larger than each of the peaks 1111 1115, 1117, 1119. In this situation, both peak 1113 and peak 1115 are discarded from the calculation. As a specific nonlimiting example, if a peak is 50% or greater than a subsequent peak, then both peaks are discarded. In another specific nonlimiting example, if a peak is 100 mL/s greater than the subsequent peak, then both peaks are discarded.

Additionally, more than one threshold may be used with combination with other measures to validate the peaks. As a specific nonlimiting example, a peak under evaluation may be required to have a minimum of 10 mL/s of magnitude and be at least 10% of the size of the previous peak, and/or, the peak may be required to have 5 mL/s of magnitude and be 20% of the size of the previous peak.

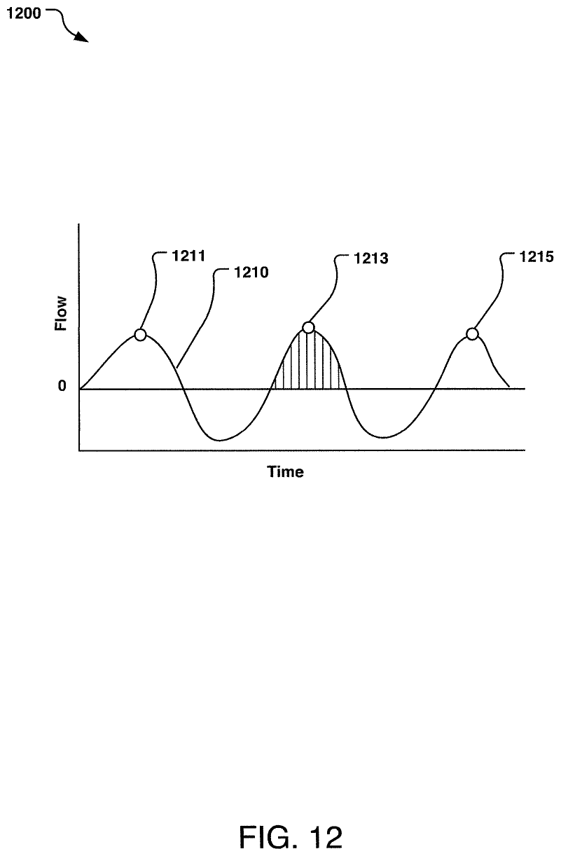

In yet another validation technique (aka, “integral around peak”), the integral of the flow around a peak is calculated and compared to the integrated subsequent peak. The relationship between the two integrals can be used to discard one or both peaks, the relationship based on a ratio of the areas, the absolute value of the areas, or another comparison. FIG. 12 has a graph 1200 with a flow signal 1210 having peaks 1211, 1213, 1215. The (greater than 0) area under the signal 1210 around the peak 1213 is calculated, as well as the area under the signal 1210 at either or both peaks 1211, 1215.

In another validation technique (aka, “number of zero flow crossings”), the number of times the flow signal crosses the axis, i.e., where flow=0, in a given time interval is used.

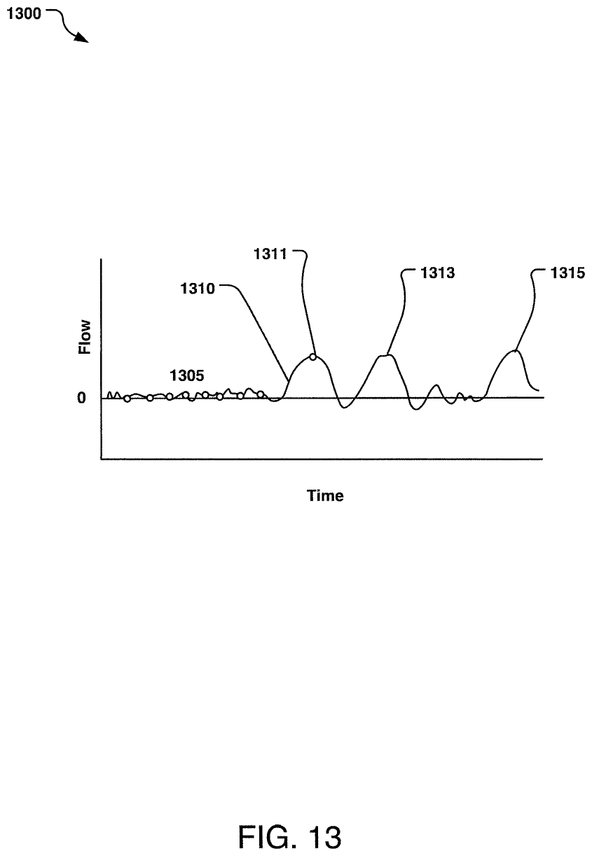

In FIG. 13, a graph 1300 has a flow signal 1310 that has a period 1305 before a peak 1311 where the number of intersections across the axis (where flow=0) is above a predetermined threshold. If the number of intersections is above the threshold, the peak 1311 is discarded. In alternate embodiments, if the number of intersections between two peaks (e.g., peak 1313 and peak 1315 is more than one, the one or both peaks 1313, 1315 are discarded. In a specific nonlimiting example, if the number of intersections is 5 or more intersections+/−1 second around a peak, the peak is discarded.

Alternatively, the number of intersections divided by the region evaluated (i.e., intersections per second) can be used, with a similar threshold to validate or discard a peak. As a nonlimiting example, a threshold of 1.2 intersection per second can be used.

The breath validation may be performed using one, all, or a subset of the techniques described above. Conditions may be applied before deciding to execute each of the checks; for example, the flow range between peaks may be performed only if the average amplitude of the flow signal in the last 15 seconds is below a threshold (e.g., 20 mL/s).

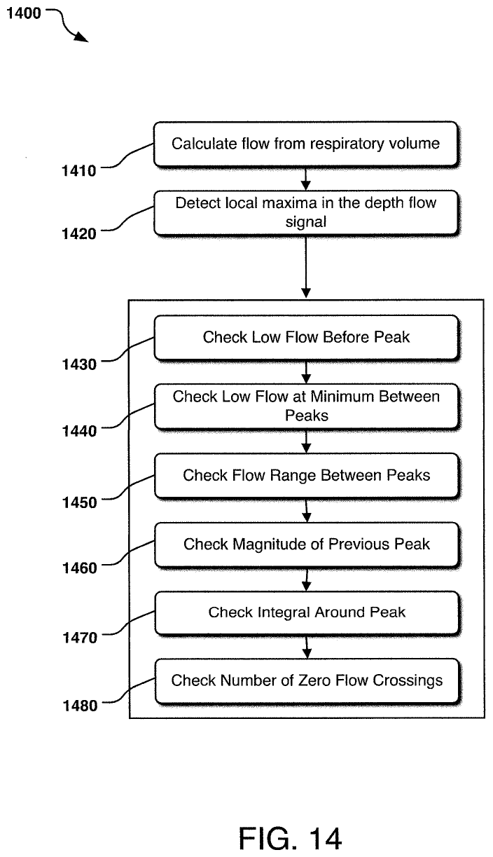

A possible method 1400, which runs all the validation checks described above is depicted in FIG. 14. It is to be understood that less than all the validation checks may be used.

In a first step 1410, the respiratory volume is acquired and the flow is calculated therefrom. A local peak maxima in the flow signal is detected in step 1420. A series of validation techniques is applied to this detected maxima in steps 1430 through 1480.

In step 1430, it is checked whether there is a low flow before the detected peak, e.g., as described above. In step 1440, it is checked whether there is a low flow at the minimum between peaks, e.g., between the detected peak and a subsequent peak, e.g., as described above. In step 1450, it is checked whether the flow range between two consecutive peaks is within a predetermined value, e.g., as described above. In step 1460, it is checked whether the magnitude of the peak immediately prior to the detected peak is within a predetermined threshold, e.g., as described above. In step 1470, the integral of the detected peak is determined, e.g., as described above. In step 1480, the number of axis crossings (where flow=0) is checked, e.g., as described above.

Any of all of the steps 1430 through 1480 may be done sequentially in any order, including one or more being done concurrently. If a discard criteria is met in any of steps 1430 through 1480, the subsequent steps are no longer applied since the peak has already been discarded. In other words, if one of the validation techniques indicates the peak should be discarded, it is not necessary to apply additional techniques.

In yet another validation technique, a neural network can be trained to determine if a peak is acceptable based on the techniques described above. For example, the above-described techniques (referred to as “flow before peak”, “flow at minimum between peaks”, “flow range between peaks”, “magnitude of previous peak”, “integral around peak” and “number of zero crossings”) can be used to train a neural network to qualify the breath, with the network used to predict if the peak should be discarded or validated. Other suitable classification models could be used, such as a decision tree, kNN algorithm (K-nearest neighbors), AdaBoost, Random Forest, etc.

As shown above, and in other validation techniques, the original waveforms (e.g., flow and/or volume, over time) can be processed to clean unwanted noise from the original signal waveform, the noise due to, e.g., movement or inherent noise from the depth camera. The original waveform or these processed waveforms can be converted to a power spectrum or power waveform.

In a real-time algorithm, the signal of interest is processed inside one or more windows of signal, each window having a duration of, e.g., 15 seconds, 30 seconds, or 60 seconds.

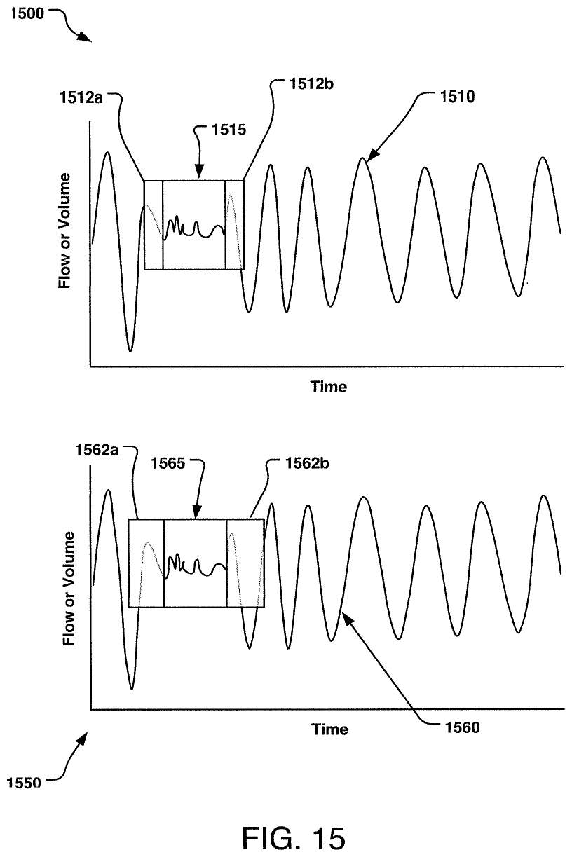

In one example technique, the cleaning of the waveform in a window is done by looking at windows where the flow has been indicated as having motion, and setting those values as being invalid; these waveforms may be flow or volume. These flagged regions containing invalid samples or data points may be further extended (to the left and/or right along the timeline) by an additional amount, e.g., 2.0 seconds. In FIG. 15, the top graph 1500, shows a sample of a waveform 1510 with a flagged window 1515 and extended regions 1512a, 1512b.

The duration of these extended regions 1512 may be a function of the size of the window 1515, signal type or other such parameters such as window duration, and may be, e.g., at least 0.5 second, at least 1 second, no more than 5 seconds. As an example, if processing a volume signal, the extension 1512 may be 3.0 seconds. In another example, if the window size is 20 seconds, the extension 1512 may be 2.0 seconds, but if the window is 60 seconds, the extension 1512 may be 6.0 seconds. In FIG. 15, the bottom graph 1550, shows a waveform 1560 with a flagged window 1565 and extended regions 1562a, 1562b, which extend farther than the extended regions 1512 of the top graph 1500.

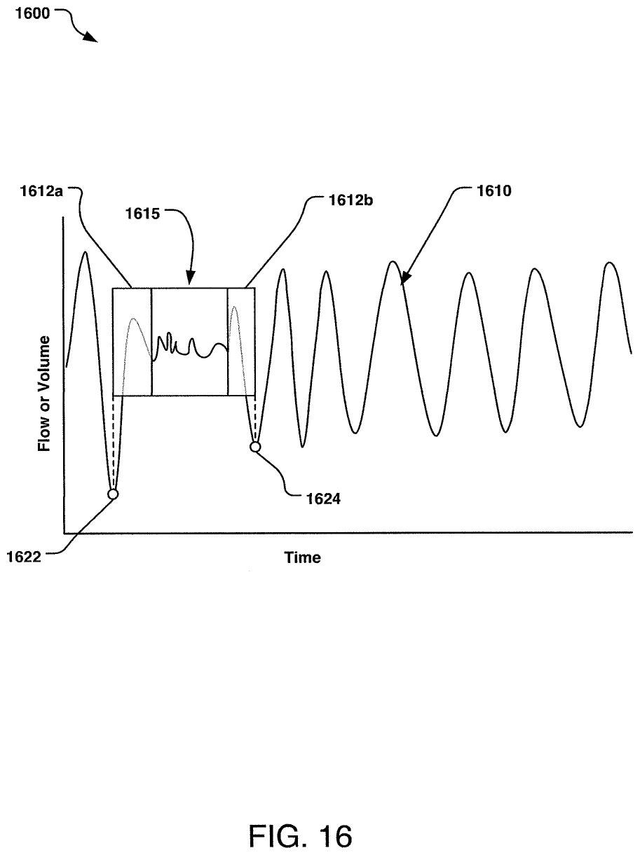

The flagged region expansions 1512, 1562 could be performed until a minimum or trough in the waveform is detected, as shown in FIG. 16. In FIG. 16, a graph 1600 shows a sample of a waveform 1610 having a window 1615 and the expansion regions 1612a, 1612b extended to align with the troughs 1622, 1624, respectively, of the waveform 1610. The extending of the expansion regions 1612 decreases the effect of spectral leakage. This removes the samples inside non-complete periods of the waveform 1610 and improves the signal quality for a subsequent power spectrum determination.

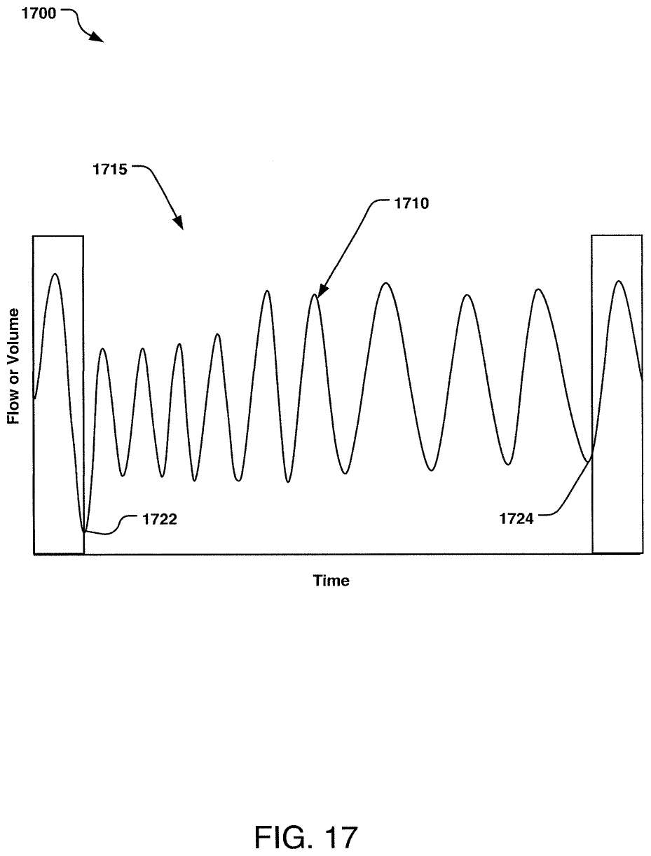

In another example technique, the signal prior to the first trough in a window is removed, as well as the signal after the last trough in the window. FIG. 17, in graph 1700, has a sample of a waveform 1710 (e.g., either flow or volume) having a first trough 1722 and a last trough 1724 in a window 1715, with numerous troughs and peaks therebetween. By removing the end troughs 1722, 1724, the non-complete periods of the waveform 1710 are removed.

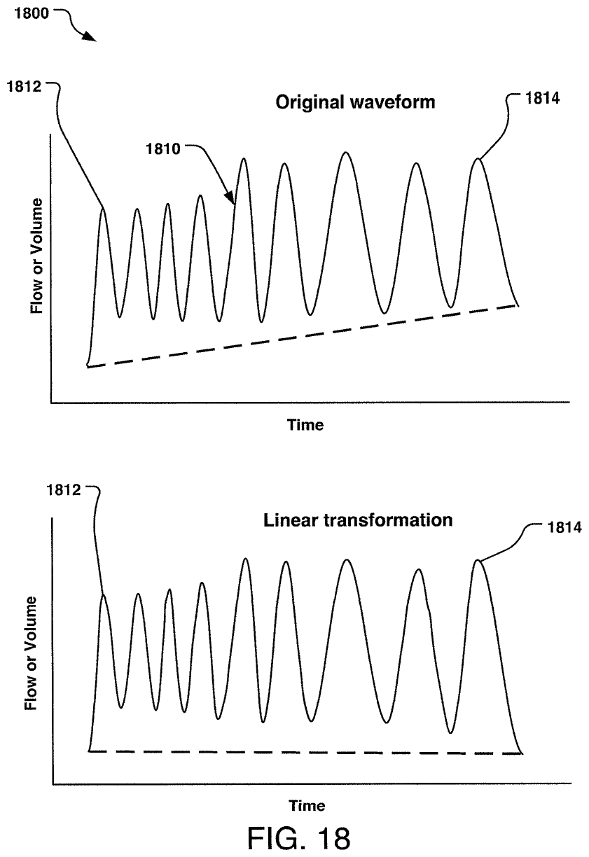

To remove the discontinuity in the peaks between the start and end of the window, a linear adjustment may be performed to the waveform signal in the window. FIG. 18 shows a sample of a waveform 1810 having a linear compensation applied to the waveform 1810, proportional to the difference in the peaks between the first and last peaks. The waveform 1810 has a first peak 1812 and a last peak 1814, with numerous periods, each having a peak, therebetween. Each sample or data point in the waveform 1810 is corrected by an amount equal to:

$$i * ( value\_end - value\_start ) / ( N - 1 ) \qquad [ 1 ]$$

For this technique, the samples or data points are indexed from 0 to N−1, where “N” is the total number of samples or data points in the window. Their index is represented in the equation by “i.” This technique improves the effect of spectral leakage and the quality of the power spectrum density.

In some calculations, a minimum number of consecutive samples or data points are present prior to the extension of a flagged region (e.g., a flagged region having invalid data points); that is, there are at least 10 samples or data points, or, e.g., 1.0 seconds worth of samples or data points in the waveform before applying the extension. This minimum number of samples may also be a function of the window size, signal type or other such parameters.

In another technique, a percentile of the samples or data points with high/low values (e.g., above the first percentile and below the 99th percentile) can be excluded from the power spectrum calculation. This technique removes potential outliers that would affect the power spectrum calculation. Other robust measures of outlier detection can be used to invalidate potential outliers.

For example, the value of (e.g.,) a flow and/or volume above and/or below fixed values can be set to be invalid; for example, if the flow is above 100 mL/s or below-100 mL/s, such samples may be invalidated and thus discarded. Such a technique is particularly suitable for high respiratory rates having lower flow values.

The previous techniques have been directed to utilizing the flow signal, derived from the volume, to detect the breaths and thus determine the respiratory rate; these techniques are particularly useful for situations with a low respiratory rate and/or high volumes. Any or all of the techniques described above may be applied in any order to the flow signal waveform, including simultaneously. For example, the order to validation may be: removal of outliers followed by the removal by the hard limit, followed by the expansion. If a discard criteria is met by any of the validation techniques, the subsequent techniques are no longer applied since the peak has already been discarded. In other words, if one of the validation techniques indicates the peak should be discarded, it is not necessary to apply additional techniques.

These processed waveforms can be further processed. In the discussion below, the processing of the signals can be used with the Lomb-Scargle method for the calculation of the power spectrum, as this method works well even if some data points are missing from the signal waveform.

In some respiratory regimes, a spectral method, such as power spectrum or power spectrum density, also referred to as a frequency spectrum or domain, can be used to validate the respiratory rate, with the respiratory rate frequency being determined by the frequency of the highest peak in the spectrum. A spectral method is particularly suitable when the respiratory rate is fast (e.g., above 20 brpm, above 25 brpm, above about 30 brpm, above about 35 brpm, above 40 brpm, above 45 brpm) and/or tidal volume is small (e.g., 50 100, 150, 200, 250 milliliters), and SNR (signal to noise ratio) is low (e.g., 0.5 (where the amplitude signal is 50% of the amplitude of the noise), 1.0 (where the signal is the same amplitude as the noise), 1.5 (where the amplitude of the signal is 50% bigger than the amplitude of the noise)).

However, spectral methods may be less reliable at lower respiratory rates (e.g., below 20 brmp, below 25 brpm, below about 30 brpm, below about 35 brpm, below 40 brpm, etc.), as the breathing pattern may be less consistent. For these low respiratory rates, the flow signal may be more reliable.

In some embodiments, the flow signal is manipulated to enhance the frequencies of interest, as depth-based waveforms (such as those from a non-contact monitoring system as described herein) may interact with the spectral method. An example of flow signal manipulation includes calculating the power signal, or power spectrum, which is a Fourier analysis, or, the squared magnitude, of the flow signal. Other signals that can be manipulated to a spectral signal (e.g., by wavelet analysis) include transthoracic impedance (TTI), piezoelectric, pressure, and accelerometer signals. Another example of flow signal manipulation includes calculating the power density or power spectrum density.

The respiratory rate can be determined from a power spectrum by evaluating the frequency value of the peak or maximum in the power spectrum. In other words, the respiratory rate frequency is determined from the frequency of the highest peak in the power spectrum.

Once the power spectrum density has been calculated on a window of signal, various signal processing techniques can be applied to enhance the peaks and frequencies for the purpose of detecting the respiratory rate frequency.

Additionally, signal processing can decrease the power of spurious frequencies that may be associated with noise from the original depth signal and enhance the signal of frequencies associated with respiration.

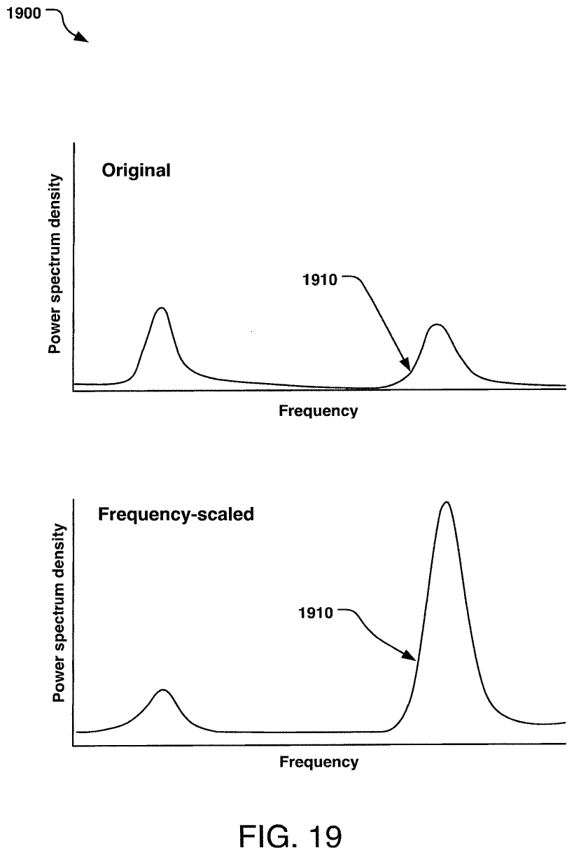

In one technique, a linear scaling of the power as a function of the frequency is applied to reduce the relative power of lower frequencies versus higher frequencies. Physiologically, the higher frequencies are expected to have lower power, which could act as a confounder for the highest peak determination. FIG. 19, in graph 1900, shows a sample of a power spectrum 1910 having a linear frequency scaling applied to the power spectrum 1910, where the top graph is the original signal and the lower graph is the frequency-scaled signal. The linear scaling would be a transformation of:

$$P ( f ) = P ( f ) \times ( a + b \times f ) \qquad [ 2 ]$$

where “P” represents the power spectrum density, “f” represents the frequency at a certain power (e.g., in units of breaths per minute (brpm)), “a” is a 0-th order coefficient and “b” is a 1st order coefficient.

Values for “a” and “b” can be static (e.g., a=1 and b=5) or may be adjusted dynamically depending on the parameters of the input signal window. For example, this could be carried by setting “a” and “b” as a function of the total power, or as a function of the frequency of the highest peak.

Note that nonlinear enhancements may also or alternately be carried out instead of the linear enhancement described by equation 1.

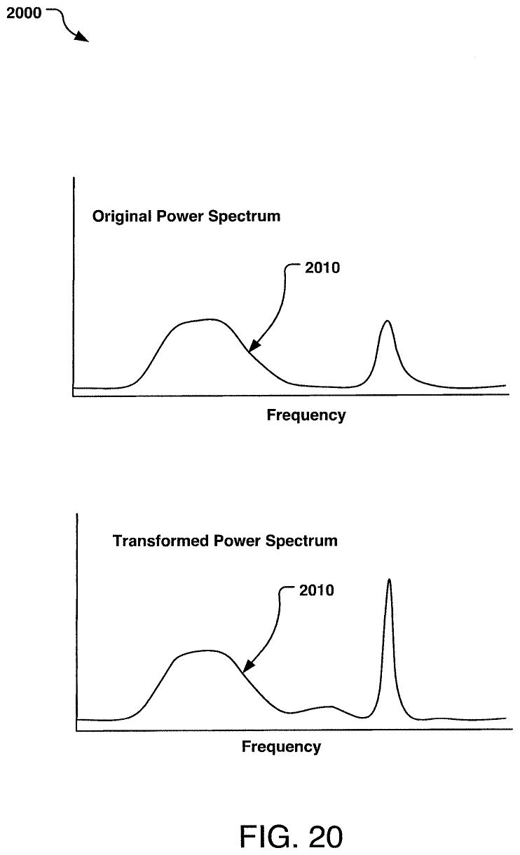

In another technique, a second derivative transformation enhancement is performed on the power spectrum density values. FIG. 20, in graph 2000, shows a sample from a power waveform 2010 having a derivative-enhanced transformation applied to the waveform 2010, where the top graph is the original signal and the lower graph is the transformed signal. The transformation achieves more definition around sharper peaks relative to flat peaks and reduces the power of the signal around spurious peaks that may appear due to noise or artifact. A second derivative enhancement can be defined as a transformation defined by:

$$P [ f ] = P [ f ] + k {P}^{′′} [ f ] \qquad [ 3 ]$$

where “k” is a scalar that controls the amount of second derivative enhancement, and “P” [f]” is the second derivate (d²P/df²) of the power spectrum (P[f]).

In an alternate technique, an additional derivative term may be used, e.g., a fourth order term for a transformation defined by:

$$P [ f ] = P [ f ] + {kP}^{′′} [ f ] + k 2 {P}^{′′′′} [ f ] \qquad [ 4 ]$$

This additional derivative term provides a smoother adjustment of the peak enhancement.

Because techniques utilizing the power spectrum may be less reliable at lower respiratory rates, as the breathing pattern may be less consistent, it may be desired to decide whether to use a power spectrum-based evaluation or a classic approach.

The respiratory rate derived from the power spectrum may work in parallel or as an alternative to identifying individual breaths directly in the respiratory waveform or in the volume or flow waveform. A real-time algorithm can be used to determine whether to use one method over the other, or when to use a combination of both methods. For example, a preliminary respiratory rate can be determined from the volume or flow waveform and from the power spectrum, and dependent on the preliminary respiratory rates, one or a combination of the waveform and the power spectrum are used to determine an accurate respiratory rate. As another example, several features of the power spectrum can be evaluated to determine if the calculation from the power spectrum is sufficiently confident.

As indicated above, the power spectrum is the squared magnitude of the Fourier transform of the corresponding flow signal. The power spectrum signal has features that are beneficial to determining an accurate respiratory rate: the power density of the greatest peak; the total power of the greatest peak (which is the integral of the power spectrum density around the peak); the total power of the power spectrum density (i.e., its integral) over a range of frequencies (e.g., from 1 brpm to 60 brpm, or, to 180 brpm for neonates); the total power of the second greatest peak in the power spectrum; the ratio between the power of the first peak and the power of the second peak; the ratio between the power density at the first peak over the power density of the second peak; and the relative peak power (i.e., the peak power divided by the total power in the power spectrum). These features may be obtained from a processed power spectrum (as explained in the previous sections), original unprocessed power spectrum, or both.

Features may also be taken directly from the input signals (flow and/or volume), like the minimum/maximum values, range, percentiles, robust mean, etc. to determine an accurate respiratory rate, as described above.

In a particular technique, the non-contact monitoring system (e.g., the system 100, 200, 400, 500, 600) can display the respiratory rate determined via the power spectrum if certain conditions are met. As examples, the power spectrum can be used when: the total power of the greatest peak is bigger than a threshold (min_power_peak); the total power of the power spectrum is within certain limits (e.g., between min_total_power, max_total_power); the total power of the greatest peak in the power spectrum is above a certain value (min_power_peak); the total power of the greatest peak in the power spectrum is below a certain value (max_power_2^{nd}_peak); the ratio between the power of the first and second peak is above a certain value (ratio_power_peaks); or the relative peak power is above a certain threshold (min_relative_power).

An estimated respiratory rate from the power spectrum may be stored for multiple samples (e.g., every second, several per minute) prior to being confirmed an accurate rate. For example, a set or minimum number of consecutive samples (e.g., 2, 3, 5, etc.) can be compared to confirm accuracy. In one example, if the samples are within a threshold (e.g., percentage or breaths per minute (brpm)), one or more of the sample values can be reported as an accurate respiratory rate (e.g., the first sample value, the last sample value, the middle or center sample value, or a mean sample value can be used). In another example, if the samples are sufficiently consistent, having, e.g., a small range or small standard deviation (e.g., a change of less than 6 brpm over the past 5 seconds), one or more of the sample values can be reported as an accurate respiratory rate. In some techniques, the respiratory rate from the power spectrum is not considered if it reports a value lower than a predetermined threshold, for example, 20, 25, 30 breaths/min.

In some techniques, a machine learning model can be trained using one or any of the features mentioned above to determine if a respiratory rate from the power spectrum is accurate and may be preferred over one calculated from the flow or volume. Example models include a decision tree, a neural network, kNN algorithm, etc.; any classification model would be appropriate.

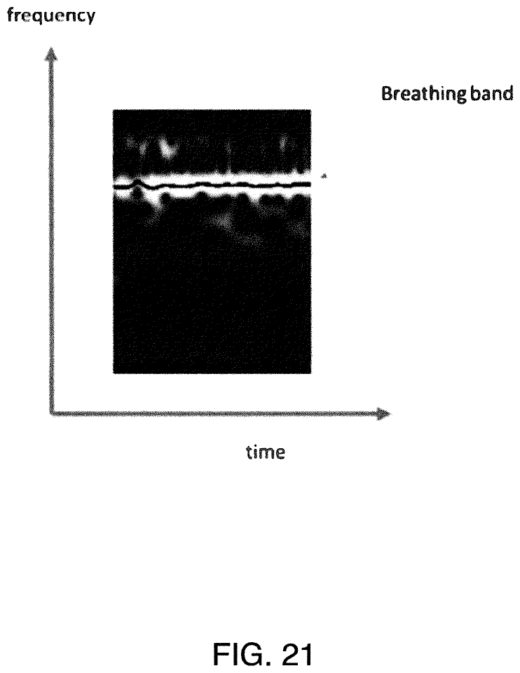

In some techniques, the window to produce the power spectrum may slide along the flow signal from which the power signal is obtained, thus producing a time-frequency representation of the flow signal. This representation corresponds to respiration and the frequency of respiration may be read directly off the peak of this band. In such a band, even very faint modulations will form distinctly coherent bands in the time frequency domain; identifying those in a single power spectrum may prove challenging. FIG. 21 illustrates the band of a wavelet time-frequency power spectrum for a respiratory rate of 40 brpm. The breathing band is clearly seen in the figure and the peak value at every time point highlighted by the line across the band indicates the frequency of respiration. Other time-frequency methods like spectrograms can also be used to identify the respiratory rate.

Further, respiratory rates obtained from different algorithms, whether a direct signal (e.g., volume, or flow) or a manipulated signal (e.g., power spectrum), can be combined to provide a composite rate. For example, a temporal derived respiratory rate can be combined with a power spectrum respiratory rate; weighting of each can be based upon metrics associated with the certainty of each method. Metrics may include the respiratory rate itself; for example, if the respiratory rate is deemed to be low, then the temporal method respiratory rate may be weighted more than the power spectrum respiratory rate, or vice versa.

The techniques and methodologies described herein provide accurate respiratory rate determinations.

A study was performed comparing respiratory rates of 30 volunteer test subjects as determined by depth measurements with the techniques described herein to respiratory rates determined the waveform of an FDA cleared End Tidal Carbon Dioxide (EtCO₂) monitor (commercially available from GE under the designation Datex-Ohmeda).

After a baseline data set at the volunteer test subject's natural respiratory rate, a range of stable respiratory rates were elicited from each subject to test the measurable range of the device. The rates were approximately 5, 10, 15, 20, 25, 30, 35, 40, 45, and 50 brpm, with some natural variation from these exact numbers and tailored to the subject's capabilities as some subjects were not able to breathe at the lower and higher respiratory rates. A paced breathing monitor was used to assist in keeping the subject's breath rate consistent and stable. Once stable breathing at the specified rate was achieved, data was collected by both the non-contact monitoring system and the Reference EtCO₂ monitor for one to three minutes per respiratory rate plateau.

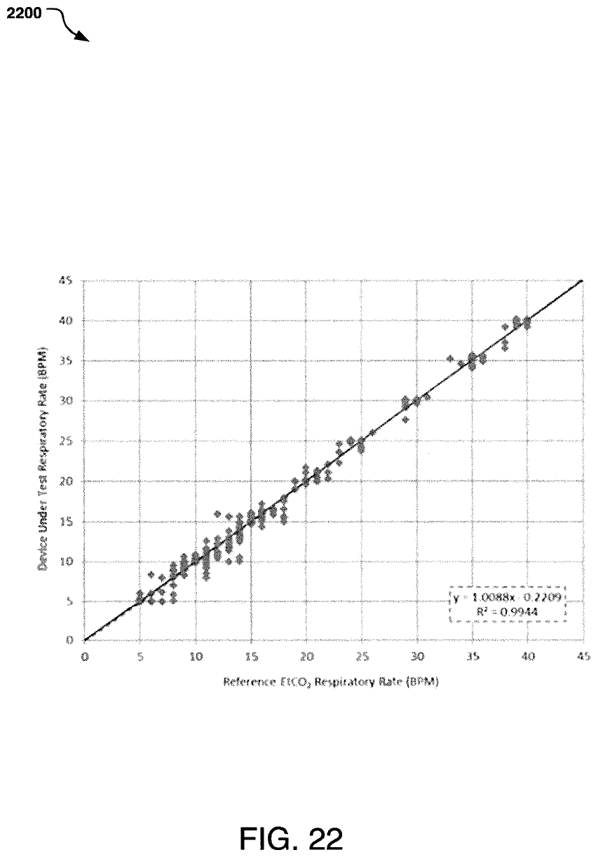

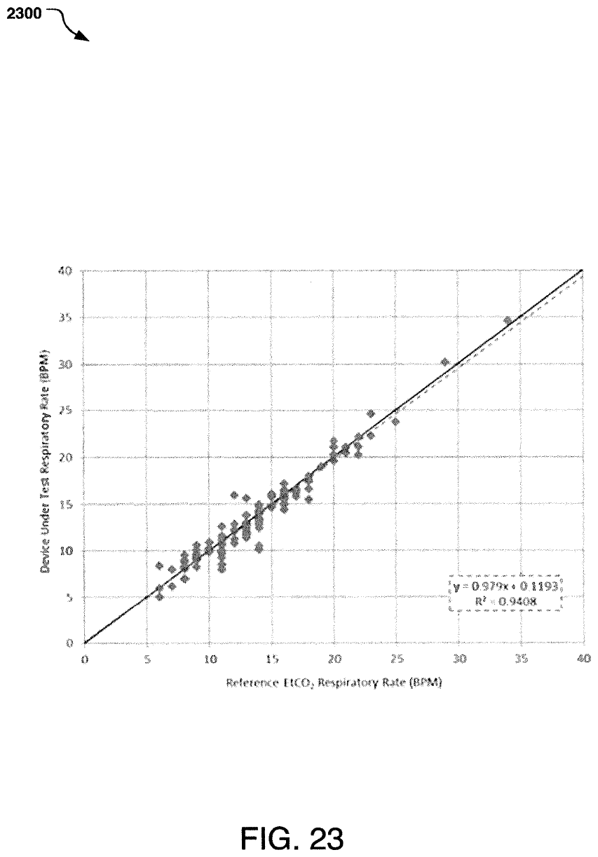

FIG. 22 in graph 2200 shows the comparison of the elicited respiratory rates as measured by the non-contact monitoring system and the Reference EtCO₂. FIG. 23 in graph 2300 shows a comparison of natural respiratory rates as measured by the non-contact monitoring system and the Reference EtCO₂, when the subject was not breathing at a desired rate or pre-set breathing rate, but rather was allowed to breath naturally.

Any or all of the respiratory rate signals determined or derived from the methodologies described herein can be visually provided to a user (e.g., clinician, caretaker, etc.) on a display, such as the displays 122, 222 of the systems 100, 200, respectively.

The above specification and examples provide a complete description of the structure, numerous methods and techniques, and use of exemplary embodiments of the invention. The above description provides specific embodiments and techniques. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A non-contact monitoring system for determining a respiratory rate of a subject, the system comprising:
a depth-sensing camera;
a display; and
a processor and a memory storing instructions therein that, when executed by the processor, cause the processor to:
determine a respiratory volume of the subject with the non-contact monitoring system using depth measurements;
calculate a respiratory flow waveform from the respiratory volume over a time period;
determine a flow-based respiratory rate from the respiratory flow waveform;
calculate a power spectrum from the respiratory flow waveform and determine a spectrum-based respiratory rate from the power spectrum;
select between the flow-based respiratory rate and the spectrum-based respiratory rate based on a respiratory rate threshold; and
show the selected respiratory rate on the display.

2. The system of claim 1, wherein the processor selects the spectrum-based respiratory rate dependent on either or both the spectrum-based respiratory rate and the flow-based respiratory rate is above the respiratory rate threshold.

3. The system of claim 1, wherein the processor selects the flow-based respiratory rate dependent on either or both the spectrum-based respiratory rate and the flow-based respiratory rate is below the respiratory rate threshold.

4. The system of claim 1, wherein the processor select both the flow-based respiratory rate and the spectrum-based respiratory rate and provides a composite respiratory rate as the selected respiratory rate.

5. The system of claim 1, wherein the respiratory rate threshold is about 30 breaths/minute.

6. The system of claim 1, wherein the processor calculates the power spectrum by utilizing a Lomb-Scargle method.

7. The system of claim 1, wherein the processor manipulates the respiratory flow waveform prior to the processor determining the flow-based respiratory rate.

8. The system of claim 7, wherein the processor manipulates the waveform by:
detecting a local maxima in a window of the waveform;
analyzing the waveform in relation to the local maxima, and
determining whether or not to discard the local maxima.

9. The system of claim 1, wherein the processor manipulates the power spectrum prior to the processor determining the spectrum-based respiratory rate.

10. The system of claim 9, wherein the processor manipulates the power spectrum by applying a linear transformation to the power spectrum, a frequency scaling to the power spectrum, or a second derivative transformation to the power spectrum.

11. A non-contact monitoring system for determining a respiratory rate of a subject, the system comprising:
a depth-sensing camera;
a display; and
a processor and a memory storing instructions therein that, when executed by the processor, cause the processor to:
determine a respiratory volume of the subject with the non-contact monitoring system using depth measurements;
calculate a respiratory flow waveform from the respiratory volume over a time period;
determine a respiratory rate from the respiratory flow waveform;
dependent on the respiratory rate from the respiratory flow waveform being above a threshold, calculate a power spectrum from the respiratory flow waveform and determine a respiratory rate from the power spectrum;
dependent on the respiratory rate from the power spectrum being determined, show on the display the respiratory rate from the power spectrum on the display for a user,
otherwise, display the respiratory rate from the respiratory flow waveform.

12. The system of claim 11, wherein the processor calculates the power spectrum from the respiratory flow waveform and determines the respiratory rate from the power spectrum dependent on the respiratory rate from the respiratory flow waveform being above 90% of the threshold.

13. The system of claim 11, wherein the processor manipulates the respiratory flow waveform prior to the processor determining the respiratory rate from the respiratory flow waveform.

14. The system of claim 11, wherein the processor manipulates the power spectrum prior to the processor determining the respiratory rate from the power spectrum.

15. A method for determining an accurate respiratory rate, the method comprising:
determining a respiratory volume of a subject with a non-contact monitoring system using depth measurements;
calculating a respiratory flow waveform from the respiratory volume over a time period;
determining a flow-based respiratory rate from the respiratory flow waveform;
calculating a power spectrum from the respiratory flow waveform and determining a spectrum-based respiratory rate from the power spectrum;
selecting between the flow-based respiratory rate and the spectrum-based respiratory rate based on a respiratory rate threshold; and
displaying the selected respiratory rate to a user.
